(19) United States
(12) Patent Application Publication
(10) Pub. No.: US 2007/0098644 A1
(43) Pub. Date: May 3, 2007

(54) Delivery of a combination therapy for asthma and chronic obstructive pulmonary disease

(75) Inventors: Jay Richard Ray II, Montgomery, TX (US); Charles David Hodge, Cypress, TX (US)

Correspondence Address: AKERMAN SENTERFITT, P.O. BOX 3188, WEST PALM BEACH, FL, 33402-3188, US

(73) Assignee: Richie's Pharmacy and Medical Supply, Incorporated, Montgomery, TX (US)

(21) Appl. No.: 11/263,723

(22) Filed: Oct. 31, 2005

(51) Int. Cl. A61K 9/12; A61K 31/56; A61K 31/16; A01N 45/00; A01N 37/18

(52) U.S. Cl. 424/45; 514/171; 514/625

(57) ABSTRACT

A method of delivery of a combination therapy to the pulmonary system that includes providing a nebulizer and a fluid comprising a long-acting corticosteroid, a long-acting beta-agonist, and a long-acting anticholinergic in a pharmaceutically acceptable vehicle, and administering the solution to the patient using the nebulizer. The corticosteroid is budesonide, the beta-agonist is formoterol and the anticholinergic is tiotropium in a an aqueous solution, suspension or emulsion suitable for administration with the nebulizer.

### FIELD OF THE INVENTION

The present invention is directed to the delivery of a combination drug therapy for asthma and chronic obstructive pulmonary disease.

### BACKGROUND OF THE INVENTION

A combination of a long-acting corticosteroid and a long-acting beta-agonist has been available for years for the treatment of asthma and chronic obstructive pulmonary disease, commonly abbreviated as COPD, such as emphysema and chronic bronchitis. Particularly, the combination of budesonide, a long-acting corticosteroid, and formoterol, a long-acting beta-agonist, is available under the brand name Symbicort® and is recommended by the National Asthma Education and Prevention Program of the National Institute of Health for long-term control and prevention of symptoms of moderate and severe persistent asthma. The combination is offered in a dry powder inhaler device marketed as Turbuhaler® by AstraZeneca.

Formoterol, a beta-agonist directly stimulates the lungs to open by binding to beta-receptor sites on smooth muscle. It is used as a rescue medication in Europe; however, the only FDA-approved indication in the US is as a preventative long-acting beta agonist. The typical dose is 12 to 24 μg administered twice daily. Budesonide is a corticosteroid that prevents and decreases inflammation. It is used as an inhalation therapy to minimize the side effects associated with the oral ingestion of steroids. This long-acting steroid is not appropriate as a rescue medication. Its typical dose is 0.25 to 0.5 mg administered twice daily.

The Northeast Essex Medicines Management Committee in the United Kingdom recommends the use of tiotropium with Symbicort® for severe COPD sufferers, those with forced expiratory volume in one second of less than 30%. Tiotropium is a long-acting antimuscarinic agent, or anticholinergic. It is supplied as a capsule containing 18 μg of tiotropium in a lactose carrier for a once daily dose that is delivered via an inhaler device trademarked as the HandiHaler®. An in vitro study of the delivery of this medication under standard conditions used a flow rate of 39 L/min for 3.1 seconds to deliver 10.4 μg of tiotropium. Unfortunately, a normal elderly patient or a patient with severe CODP cannot achieve such a flow rate.

A nebulizer is a delivery device that was designed to overcome the pulmonary limitations of patients. Sometimes called a “breathing treatment,” a nebulizer creates a mist containing the drug, which makes it easy and pleasant to breath the drug into the lungs. A nebulizer requires formulations in liquid form to function properly. Nebulizers work by forcing air through a cup containing the liquid medicine. This produces tiny mist-like particles of the liquid so that they can be inhaled deeply into the airways. Other nebulizers use an ultrasonic mechanism to generate the mist.

No dosage form that combine the three agents for treating asthma or COPD has been available or described. A desired triple therapy would include a long-acting antimuscarinic agent, or anticholinergic, with the long-acting corticosteroid and a long-acting beta-agonist combination described above. All three medications are presently available commercially with the delivery mode almost exclusively that of inhaling a dry powder using an inhaler, as in the case of Symbicort® and Forabil®. Budesonide is also available to be delivered as an aqueous solution via a hand held pump. The ability to prepare a stable saline solution of dry powder formoterol and administer it via a nebulizer has been reported but formoterol is not presently marketed in this form. The sole manufacturer of Spiriva®, the brand name of tiotropium by Boehringer Ingelheim, issued a drug information letter on Feb. 8, 2005 that concluded that “this product cannot to be used in a nebulizer”. In spite of the desire to use the three drugs in combination, no description of a convenient dosage form of the drugs with a delivery method that enables a patient with a compromised pulmonary system to inhale has been realized and the ability to achieve this goal is questionable since the possibility of putting the anticholinergic in a vehicle for use with a nebulizer has been discouraged. To this end, a vehicle to deliver a combination therapy having a long-acting corticosteroid with a long-acting beta-agonist and a long-acting anticholinergic remains highly desirable for patients in need of such therapy. The method of delivery should be one that is effective for the patient that can benefit from the therapy. An improvement over delivery with an inhaler is needed.

### SUMMARY OF THE INVENTION

The present invention is directed to a method to deliver a combination therapy to the pulmonary system where a nebulizer is provided with a fluid containing a long-acting corticosteroid, a long-acting beta-agonist, and a long-acting anticholinergic in a pharmaceutically acceptable vehicle and the solution is administered to the patient using the nebulizer. A preferred corticosteroid is budesonide. A preferred beta-agonist is formoterol, and a preferred anticholinergic is tiotropium. The preferred vehicle is an aqueous solution, suspension or emulsion.

In one embodiment, the fluid contains approximately 3 to approximately 24 μg, preferably approximately 5 to approximately 7 μg, most preferably approximately 6 μg of formoterol; preferably 1.5 to approximately 15 μg, preferably approximately 3.5 to approximately 5.5 μg, most preferably at approximately 4.5 μg of tiotropium; and 0.1 to approximately 0.6 mg, preferably approximately 0.2 to approximately 0.3 mg, most preferably 0.25 mg of budesonide per 2 mL of the fluid. The fluid may also contain approximately 0.01 to approximately 0.04 mL, preferably approximately 0.02 to approximately 0.03 mL Polysorbate 80; and approximately 50 to approximately 400 μg Trisodium EDETATE to stabilize the fluid. The vehicle can be a 0.9% sodium chloride solution. The fluid has a pH of less than approximately 8.4 and has a preferred pH of between approximately 5.2 and approximately 6.8.

The fluid is packaged in vials such that one, two or more vials can be used to achieve the prescribed dosage where the contents of the vials are used sequentially or are combined into the nebulizer for administration in a single dosage session.

The invention is also directed to a pharmaceutical composition that is an aqueous solution, suspension or emulsion having a mixture of effective amounts of formoterol, budesonide, and tiotropium in any physiologically acceptable salts of these medications such that the composition is suitable for delivery by inhalation for the treatment of asthma and COPD.

The composition provides tiotropium as tiotropium bromide and formoterol as formoterol fumarate. The ranges of the amount of these drugs include formoterol at approximately 3 to approximately 24 μg, preferably approximately 5 to approximately 7 μg, most preferably approximately 6 μg per 2 mL of aqueous solution, suspension or emulsion. Tiotropium may be included at approximately 1.5 to approximately 15 μg, preferably approximately 3.5 to approximately 5.5 μg, most preferably at approximately 4.5 μg per 2 mL of aqueous solution, suspension or emulsion. Budesonide at approximately 0.1 to approximately 0.6 mg, preferably approximately 0.2 to approximately 0.3 mg, most preferably 0.25 mg per 2 mL of aqueous solution, suspension or emulsion. To achieve this dosage form, approximately 0.01 to approximately 0.04 mL, preferably approximately 0.02 to approximately 0.03 mL Polysorbate 80, approximately 50 to approximately 400 μg Trisodium EDETATE, and approximately 9 to approximately 30 mg, preferably approximately 15 to approximately 20 mg, and most preferably approximately 18 mg of sodium chloride per 2 mL of aqueous solution, suspension or emulsion. It will be appreciated that Polysorbate is not required, and that the dosage form may be achieved using sterile water. This aqueous solution, suspension or emulsion has a pH of less than 8.4 and preferrably has a pH of 5.2 to 6.8. This composition is suitable for delivery by inhalation using a nebulizer.

### DETAILED DESCRIPTION OF THE INVENTION

The present invention is directed to an effective treatment of asthma or chronic obstructive pulmonary disease such as emphysema and chronic bronchitis. Treatment involves the delivery of the needed drug to the pulmonary system. The drugs delivered to the lungs are of three types: a beta-agonist to stimulate beta-receptors in the autonomic nervous system to open the airways by relaxing the muscles around the airways that may tighten during bronchospasms and relieve dyspnea; a corticosteroid to reduce or prevent inflammation; and an anticholinergic, specifically an antimuscarinic agent, to operate on the muscarinic acetylcholine receptors reducing the effects mediated by acetylcholine in the nervous system and acting as a bronchiodilator. In some instances, the desired dosage form is intended for use by patients with severe conditions. The invention is also directed to a regimen of dosing that maintains the appropriate levels of the drugs and is administered in a form that a patient with a weakened condition can achieve the intended dosage during a single delivery session.

The required drugs can be relatively long-acting such that delivery of the drug does not require an unreasonable regimen of the patient with respect to the portion of the day which must be dedicated to the delivery of the therapy. The drugs must also be compatible with each other. A vehicle by which they may be mixed and co-administered is required. A triple combination that achieves these goals is that of: formoterol, budesonide, and tiotropium, the beta-agonist, corticosteroid, and anticholinergic, respectively. The choice of these drugs achieves the goal for a minimally inconveniencing of the patient. In at least one embodiment, the dosages of these medications can be packaged for administration only twice daily. The most desired vehicle is water but can include alcohols or other co-solvents or any combination thereof. Buffers or other components to adjust and control the pH and metal complexing agents to enhance the miscibility of the active components can be included in the formulation. Other ingredients can be included to adjust other properties of the solution such as viscosity and emulsion stability while maintaining the desired chemical compatibility and stability of the mixture.

Formoterol is the common name for rel-N-[2-Hydroxy-5-[(1R)-1-hydroxy-2-[[(1R)-2-(4-methoxyphenyl)-1-methylethyl]amino]ethyl]phenyl]formamide with the molecular formula C₁₉H₂₄N₂O₄ and is normally provided as the fumarate dihydrate in powder form. The molecular formula of the fumarate salt is (C₁₉H₂₄N₂O₄)₂.C₄H₄O₄.2H₂O. Budesonide is the common name for (11β,16α)-16,17-[Butylidenebis(oxy)]-11.12-dihydroxypregna- 1,4-diene-3,20-dione with the molecular formula C₂₅H₃₄O₆. Tiotropium is provided as tiotropium bromide which is a common name for (1α,2β,4β,5α,7β)-7-[(hydroxydi-2-thienylacetyl)oxy]-9,9-dimethyl-3-oxa-9-azoniatricyclo[3.3.1.0^{2.4}]nonane bromide with molecular formula C₁₉H₂₂BrNO₄S₂.

Patients needing this combination of pharmaceutical agents often do not have a sufficient physical condition, particularly in their pulmonary system, to achieve the desired dose of some of these medications, particularly the tiotropium, when used individually in inhalers. The present invention is directed to overcoming the limitations of the inhalation methods available. The drugs are combined as an aqueous solution for delivery by a nebulizer. There are several advantages to the use of a nebulizer for medications. In some embodiments, the primary advantage is that its use requires only simple tidal breathing to receive the designed dose of the pharmaceutical. Although literature by the manufacturer of tiotropium has reported that it is inappropriate to use a nebulizer with their product, it has been discovered that the preparation of a mixture of these medications in an aqueous solution is possible.

An exemplary dosage form for delivery by a nebulizer is formulated as given below. It is designed to deliver 6 μg formoterol, 4.5 μg tiotropium and 0.25 mg budesonide when 2 mL of the aqueous solution is used with a nebulizer. Although many different commercially available nebulizers may be used, a Pari LC Plus® with a Pari Ultra® compressor was used in for the administration in the studies leading to this application. It is useful for a therapy where two ampules are used two times a day to deliver the recommended doses of the three components. This nebulizer delivered regimen has given vastly superior results with COPD sufferers to that of the administration of the medications separately via the normal inhalers with which they are provided.

### EXAMPLE 1

Dosage Form

| | μg/vial | |
|---|---|---|
| Active Ingredient | | |
| Tiotropium | 4.5 | |
| Formoterol | 6.0 | |
| Budesonide | 250 | |
| Inactive Ingredient | | |
| Polysorbate 80 | 0.02 | mL/vial |
| Trisodium EDETATE | 200 | μg/vial |

| | |
|---|---|
| 0.9% Sodium Chloride solution | quantity sufficient |
| | up to 2 mL |
| pH | 5.2-6.8 |

To prepare the formulation above 0.501 g Tiotropium powder from capsules containing 18 μkg tiotropium/capsule is combined with 1 L of sterile sodium chloride for irrigation, 0.9% NaCl and homogenized to assure dispersion. To the dispersion is added 0.1 g Trisodium EDETATE, a complexing agent, and 10 mL of sterile Polysorbate 80 NF, a polyether emulsifier. To this suspension is added 0.125 g Budesonide, micronized which is then heated in a autoclave at 121-34° C. for 20 minutes and then stirred. To this solution is added 5 mL Formoterol 0.6 mg/mL solution through a 0.22 micron filter. The mixture is then separated, placing 2 mL in sterile vials. The pH ranges from 5.2 to 6.8 for this formulation as prepared above. The formulation can be outside of this range but cannot be greater than 8.4 to avoid degradation of ingredients, particularly the tiotropium. The pH can be adjusted using hydrochloric acid solution or sodium hydroxide solution as needed.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A method of delivery of a combination therapy to the pulmonary system comprising:
providing a nebulizer;
providing a fluid comprising a long-acting corticosteroid, a long-acting beta-agonist, and a long-acting anticholinergic in a pharmaceutically acceptable vehicle; and
administering the solution to the patient using the nebulizer.

2. The method of claim 1, wherein the corticosteroid is budesonide.

3. The method of claim 1, wherein the beta-agonist is formoterol.

4. The method of claim 1, wherein the anticholinergic is tiotropium.

5. The method of claim 1, wherein the vehicle is an aqueous solution, suspension or emulsion.

6. The method of claim 1, wherein the fluid contains 3-24 μg of formoterol, 1.5-15 μg of tiotropium and 0.1-0.6 mg of budesonide per 2 mL of the fluid.

7. The method of claim 6, wherein the fluid contains 5-7 μg of formoterol, 3.5-5.5 μg of tiotropium and 0.2-0.3 mg of budesonide per 2 mL of the fluid.

8. The method of claim 7, wherein the fluid contains 6 μg of formoterol, 4.5 μg of tiotropium and 0.25 mg of budesonide per 2 mL of the fluid.

9. The method of claim 1, wherein the fluid further comprises 0.01 to 0.04 mL Polysorbate 80 and 50 to 400 μTrisodium EDETATE.

10. The method of claim 1, wherein the vehicle is a sodium chloride solution and/or water.

11. The method of claim 1, wherein the fluid has a pH of less than approximately 8.4.

12. The method of claim 11. wherein the fluid has a pH of between approximately 5.2 and approximately 6.8.

13. The method of claim 1, wherein the fluid is packaged in vials such that one, two or more vials can be used to achieve the prescribed dosage where the contents of the vials are used sequentially or are combined into the nebulizer for administration in a single dosage session.

14. A pharmaceutical composition comprising an aqueous solution, suspension or emulsion having a mixture of effective amounts of formoterol, budesonide, and tiotropium in any physiologically acceptable salts thereof, wherein the composition is suitable for delivery by inhalation for the treatment of asthma and COPD.

15. The composition of claim 14, wherein the tiotropium is tiotropium bromide.

16. The composition of claim 14, wherein the formoterol is formoterol fumarate.

17. The composition of claim 14, wherein the amount of formoterol is 3-24 μg, the amount of tiotropium is 1.5-15 μg, and the amount of budesonide is 0.1-0.6 mg per 2 mL of aqueous solution, suspension or emulsion.

18. The composition of claim 14, wherein the amount of formoterol is 5-7 μg, the amount of tiotropium is 3.5-5.5 μg, and the amount of budesonide is 0.2-0.3 mg per 2 mL of aqueous solution, suspension or emulsion.

19. The composition of claim 18, wherein the amount of formoterol is 6 μg, the amount of tiotropium is 4.5 μg, and the amount of budesonide is 0.25 mg per 2 mL of aqueous solution, suspension or emulsion.

20. The composition of claim 19, wherein the aqueous solution, suspension or emulsion includes 42 to 42 μg Polysorbate 80, 0.4 to 0.4 μg Trisodium EDETATE and 180 to 180 mg of sodium chloride per 2 mL of aqueous solution, suspension or emulsion.

21. The composition of claim 14, wherein the aqueous solution, suspension or emulsion has a pH of less than 8.4.

22. The composition of claim 14, wherein the aqueous solution, suspension or emulsion has a pH of 5.2 to 6.8.

23. The composition of claim 14, wherein the composition is suitable for delivery using a nebulizer.
